(19) United States
(12) Patent Application Publication
(10) Pub. No.: US 2008/0207591 A1
(43) Pub. Date: Aug. 28, 2008

(54) Organic Compounds

(76) Inventors: Paul W. Manley, Arlesheim (CH); Johannes Roesel, Riehen (CH)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 11/995,602

(22) PCT Filed: Jul. 19, 2006
(86) PCT No.: PCT/EP06/64430

§ 371 Date: Jan. 14, 2008

Related U.S. Application Data
(60) Provisional application No. 60/701,103, filed on Jul. 20, 2005.

(51) Int. Cl. A61K 31/506; A61K 31/553; A61K 31/496; A61P 35/02; A61P 35/00

(52) U.S. Cl. 514/211.08; 514/275; 514/252.14

(57) ABSTRACT

The invention provides a pharmaceutical combination comprising:
a) a pyrimidylaminobenzamide compound; and
b) a Flt-3 inhibitor,
   and a method for treating or preventing a proliferative disease using such a combination.

The present invention relates to a pharmaceutical combination comprising a pyrimidylaminobenzamide compound and a Flt-3 inhibitor, and the uses of such a combination, e.g., in proliferative diseases, e.g., tumors, myelomas, leukemias, psoriasis, restenosis, sclerodermitis and fibrosis.

In spite of numerous treatment options for proliferative disease patients, there remains a need for effective and safe antiproliferative agents and a need for their preferential use in combination therapy.

### SUMMARY OF THE INVENTION

It has now been found that a combination comprising at least one pyrimidylaminobenzamide compound and a Flt-3 inhibitor, e.g., as defined below, has a beneficial effect on proliferative diseases, e.g., tumors, myelomas, leukemias, psoriasis, restenosis, sclerodermitis and fibrosis.

### DETAILED DESCRIPTION OF THE INVENTION

The present invention relates to the use of pyrimidylaminobenzamide compounds of formula (I):

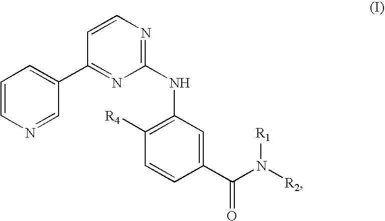

wherein
R₁ represents hydrogen, lower alkyl, lower alkoxy-lower alkyl, acyloxy-lower alkyl, carboxy-lower alkyl, lower alkoxycarbonyl-lower alkyl or phenyl-lower alkyl;
R₂ represents hydrogen, lower alkyl, optionally substituted by one or more identical or different radicals R₃, cycloalkyl, benzcycloalkyl, heterocyclyl, an aryl group, or a mono- or bicyclic heteroaryl group comprising zero, one, two or three ring nitrogen atoms and zero or one oxygen atom and zero or one sulfur atom, which groups in each case are unsubstituted or mono- or polysubstituted; and
   R₃ represents hydroxy, lower alkoxy, acyloxy, carboxy, lower alkoxycarbonyl, carbamoyl, N-mono- or N,N-disubstituted carbamoyl, amino, mono- or disubstituted amino, cycloalkyl, heterocyclyl, an aryl group, or a mono- or bicyclic heteroaryl group comprising zero, one, two or three ring nitrogen atoms and zero or one oxygen atom and zero or one sulfur atom, which groups in each case are unsubstituted or mono- or polysubstituted, or
R₁ and R₂, together, represent alkylene with four, five or six carbon atoms optionally mono- or disubstituted by lower alkyl, cycloalkyl, heterocyclyl, phenyl, hydroxy, lower alkoxy, amino, mono- or disubstituted amino, oxo, pyridyl, pyrazinyl or pyrimidinyl; benzalkylene with four or five carbon atoms; oxaalkylene with one oxygen and three or four carbon atoms; or azaalkylene with one nitrogen and three or four carbon atoms wherein nitrogen is unsubstituted or substituted by lower alkyl, phenyl-lower alkyl, lower alkoxycarbonyl-lower alkyl, carboxy-lower alkyl, carbamoyl-lower alkyl, N-mono- or N,N-disubstituted carbamoyl-lower alkyl, cycloalkyl, lower alkoxycarbonyl, carboxy, phenyl, substituted phenyl, pyridinyl, pyrimidinyl or pyrazinyl; and
R₄ represents hydrogen, lower alkyl or halogen;
   and a N-oxide or a pharmaceutically acceptable salt of such a compound for the preparation of a pharmaceutical composition for the treatment of kinase dependent diseases.

The general terms used hereinbefore and hereinafter preferably have within the context of this disclosure the following meanings, unless otherwise indicated:

The prefix “lower” denotes a radical having up to and including a maximum of 7, especially up to and including a maximum of 4 carbon atoms, the radicals in question being either linear or branched with single or multiple branching.

Where the plural form is used for compounds, salts, and the like, this is taken to mean also a single compound, salt or the like.

Any asymmetric carbon atoms may be present in the (R)-, (S)- or (R,S)-configuration, preferably in the (R)- or (S)-configuration. The compounds may thus be present as mixtures of isomers or as pure isomers, preferably as enantiomer-pure diastereomers.

The invention relates also to possible tautomers of the compounds of formula (I).

Lower alkyl is preferably alkyl with from and including 1 up to and including 7, preferably from and including 1 to and including 4, and is linear or branched; preferably, lower alkyl is butyl, such as n-butyl, sec-butyl, isobutyl, tert-butyl, propyl, such as n-propyl or isopropyl, ethyl or methyl. Preferably lower alkyl is methyl, propyl or tert-butyl.

Lower acyl is preferably formyl or lower alkylcarbonyl, in particular, acetyl.

An aryl group is an aromatic radical which is bound to the molecule via a bond located at an aromatic ring carbon atom of the radical. In a preferred embodiment, aryl is an aromatic radical having 6 to 14 carbon atoms, especially phenyl, naphthyl, tetrahydronaphthyl, fluorenyl or phenanthrenyl, and is unsubstituted or substituted by one or more, preferably up to three, especially one or two substituents, especially selected from amino, mono- or disubstituted amino, halogen, lower alkyl, substituted lower alkyl, lower alkenyl, lower alkynyl, phenyl, hydroxy, etherifled or esterifled hydroxy, nitro, cyano, carboxy, esterifled carboxy, alkanoyl, benzoyl, carbamoyl, N-mono- or N,N-disubstituted carbamoyl, amidino, guanidino, ureido, mercapto, sulfo, lower alkylthio, phenylthio, phenyl-lower alkylthio, lower alkylphenylthio, lower alkylsulfinyl, phenylsulfinyl, phenyl-lower alkylsulfinyl, lower alkylphenylsulfinyl, lower alkylsulfonyl, phenylsulfonyl, phenyl-lower alkylsulfonyl, lower alkylphenylsulfonyl, halogen-lower alkylmercapto, halogen-lower alkylsulfonyl, such as especially trifluoromethanesulfonyl, dihydroxybora (—B(OH)₂), heterocyclyl, a mono- or bicyclic heteroaryl group and lower alkylene dioxy bound at adjacent C-atoms of the ring, such as methylene dioxy. Aryl is more preferably phenyl, naphthyl or tetrahydronaphthyl, which in each case is either unsubstituted or independently substituted by one or two substituents selected from the group comprising halogen, especially fluorine, chlorine, or bromine; hydroxy; hydroxy etherifled by lower alkyl, e.g., by methyl, by halogen-lower alkyl, e.g., trifluoromethyl, or by phenyl; lower alkylene dioxy bound to two adjacent C-atoms, e.g., methylenedioxy, lower alkyl, e.g., methyl or propyl; halogen-lower alkyl, e.g., trifluoromethyl; hydroxy-lower alkyl, e.g., hydroxymethyl or 2-hydroxy-2-propyl; lower alkoxy-lower alkyl; e.g., methoxymethyl or 2-methoxyethyl; lower alkoxycarbonyl-lower alkyl, e.g., methoxycarbonylmethyl; lower alkynyl, such as 1-propynyl; esterifled carboxy, especially lower alkoxycarbonyl, e.g., methoxycarbonyl, n-propoxy carbonyl or iso-propoxy carbonyl; N-mono-substituted carbamoyl, in particular, carbamoyl monosubstituted by lower alkyl, e.g., methyl, n-propyl or iso-propyl; amino; lower alkylamino, e.g., methylamino; di-lower alkylamino, e.g., dimethylamino or diethylamino; lower alkylene-amino, e.g., pyrrolidino or piperidino; lower oxaalkylene-amino, e.g., morpholino, lower azaalkylene-amino, e.g., piperazino, acylamino, e.g., acetylamino or benzoylamino; lower alkylsulfonyl, e.g., methylsulfonyl; sulfamoyl; or phenylsulfonyl.

A cycloalkyl group is preferably cyclopropyl, cyclopentyl, cyclohexyl or cycloheptyl, and may be unsubstituted or substituted by one or more, especially one or two, substituents selected from the group defined above as substituents for aryl, most preferably by lower alkyl, such as methyl, lower alkoxy, such as methoxy or ethoxy, or hydroxy, and further by oxo or fused to a benzo ring, such as in benzcyclopentyl or benzcyclohexyl.

Substituted alkyl is alkyl as last defined, especially lower alkyl, preferably methyl; where one or more, especially up to three, substituents may be present, primarily from the group selected from halogen, especially fluorine, amino, N-lower alkylamino, N,N-di-lower alkylamino, N-lower alkanoylamino, hydroxy, cyano, carboxy, lower alkoxycarbonyl, and phenyl-lower alkoxycarbonyl. Trifluoromethyl is especially preferred.

Mono- or disubstituted amino is especially amino substituted by one or two radicals selected independently of one another from lower alkyl, such as methyl; hydroxy-lower alkyl, such as 2-hydroxyethyl; lower alkoxy lower alkyl, such as methoxy ethyl; phenyl-lower alkyl, such as benzyl or 2-phenylethyl; lower alkanoyl, such as acetyl; benzoyl; substituted benzoyl, wherein the phenyl radical is especially substituted by one or more, preferably one or two, substituents selected from nitro, amino, halogen, N-lower alkylamino, N,N-di-lower alkylamino, hydroxy, cyano, carboxy, lower alkoxycarbonyl, lower alkanoyl, and carbamoyl; and phenyl-lower alkoxycarbonyl, wherein the phenyl radical is unsubstituted or especially substituted by one or more, preferably one or two, substituents selected from nitro, amino, halogen, N-lower alkylamino, N,N-di-lower alkylamino, hydroxy, cyano, carboxy, lower alkoxycarbonyl, lower alkanoyl, and carbamoyl; and is preferably N-lower alkylamino, such as N-methylamino, hydroxy-lower alkylamino, such as 2-hydroxyethylamino or 2-hydroxypropyl, lower alkoxy lower alkyl, such as methoxy ethyl, phenyl-lower alkylamino, such as benzylamino, N,N-di-lower alkylamino, N-phenyl-lower alkyl-N-lower alkylamino, N,N-di-lower alkylphenylamino, lower alkanoylamino, such as acetylamino, or a substituent selected from the group comprising benzoylamino and phenyl-lower alkoxycarbonylamino, wherein the phenyl radical in each case is unsubstituted or especially substituted by nitro or amino, or also by halogen, amino, N-lower alkylamino, N,N-di-lower alkylamino, hydroxy, cyano, carboxy, lower alkoxycarbonyl, lower alkanoyl, carbamoyl or aminocarbonylamino. Disubstituted amino is also lower alkylene-amino, e.g., pyrrolidino, 2-oxopyrrolidino or piperidino; lower oxaalkylene-amino, e.g., morpholino, or lower azaalkylene-amino, e.g., piperazino or N-substituted piperazino, such as N-methylpiperazino or N-methoxycarbonylpiperazino.

Halogen is especially fluorine, chlorine, bromine, or iodine, especially fluorine, chlorine or bromine.

Etherifled hydroxy is especially C₈-C₂₀alkyloxy, such as n-decyloxy, lower alkoxy (preferred), such as methoxy, ethoxy, isopropyloxy, or tert-butyloxy, phenyl-lower alkoxy, such as benzyloxy, phenyloxy, halogen-lower alkoxy, such as trifluoromethoxy, 2,2,2-trifluoroethoxy or 1,1,2,2-tetrafluoroethoxy, or lower alkoxy which is substituted by mono- or bicyclic heteroaryl comprising one or two nitrogen atoms, preferably lower alkoxy which is substituted by imidazolyl, such as 1H-imidazol-1-yl, pyrrolyl, benzimidazolyl, such as 1-benzimidazolyl, pyridyl, especially 2-, 3- or 4-pyridyl, pyrimidinyl, especially 2-pyrimidinyl, pyrazinyl, isoquinolinyl, especially 3-isoquinolinyl, quinolinyl, indolyl or thiazolyl.

Esterifled hydroxy is especially lower alkanoyloxy, benzoyloxy, lower alkoxycarbonyloxy, such as tert-butoxycarbonyloxy, or phenyl-lower alkoxycarbonyloxy, such as benzyloxycarbonyloxy.

Esterifled carboxy is especially lower alkoxycarbonyl, such as tert-butoxycarbonyl, iso-propoxycarbonyl, methoxycarbonyl or ethoxycarbonyl, phenyl-lower alkoxycarbonyl, or phenyloxycarbonyl.

Alkanoyl is primarily alkylcarbonyl, especially lower alkanoyl, e.g., acetyl.

N-Mono- or N,N-disubstituted carbamoyl is especially substituted by one or two substituents independently selected from lower alkyl, phenyl-lower alkyl and hydroxy-lower alkyl, or lower alkylene, oxa-lower alkylene or aza-lower alkylene optionally substituted at the terminal nitrogen atom.

A mono- or bicyclic heteroaryl group comprising zero, one, two or three ring nitrogen atoms and zero or one oxygen atom and zero or one sulfur atom, which groups in each case are unsubstituted or mono- or polysubstituted, refers to a heterocyclic moiety that is unsaturated in the ring binding the heteroaryl radical to the rest of the molecule in formula (I) and is preferably a ring, where in the binding ring, but optionally also in any annealed ring, at least one carbon atom is replaced by a heteroatom selected from the group consisting of nitrogen, oxygen and sulfur; where the binding ring preferably has 5 to 12, more preferably 5 or 6 ring atoms; and which may be unsubstituted or substituted by one or more, especially one or two, substituents selected from the group defined above as substituents for aryl, most preferably by lower alkyl, such as methyl, lower alkoxy, such as methoxy or ethoxy, or hydroxy. Preferably the mono- or bicyclic heteroaryl group is selected from 2H-pyrrolyl, pyrrolyl, imidazolyl, benzimidazolyl, pyrazolyl, indazolyl, purinyl, pyridyl, pyrazinyl, pyrimidinyl, pyridazinyl, 4H-quinolizinyl, isoquinolyl, quinolyl, phthalazinyl, naphthyridinyl, quinoxalyl, quinazolinyl, quinnolinyl, pteridinyl, indolizinyl, 3H-indolyl, indolyl, isoindolyl, oxazolyl, isoxazolyl, thiazolyl, isothiazolyl, triazolyl, tetrazolyl, furazanyl, benzo[d]pyrazolyl, thienyl and furanyl. More preferably the mono- or bicyclic heteroaryl group is selected from the group consisting of pyrrolyl, imidazolyl, such as 1H-imidazol-1-yl, benzimidazolyl, such as 1-benzimidazolyl, indazolyl, especially 5-indazolyl, pyridyl, especially 2-, 3- or 4-pyridyl, pyrimidinyl, especially 2-pyrimidinyl, pyrazinyl, isoquinolinyl, especially 3-isoquinolinyl, quinolinyl, especially 4- or 8-quinolinyl, indolyl, especially 3-indolyl, thiazolyl, benzo[d]pyrazolyl, thienyl, and furanyl. In one preferred embodiment of the invention the pyridyl radical is substituted by hydroxy in ortho position to the nitrogen atom and hence exists at least partially in the form of the corresponding tautomer which is pyridin-(1H)2-one. In another preferred embodiment, the pyrimidinyl radical is substituted by hydroxy both in position 2 and 4 and hence exists in several tautomeric forms, e.g., as pyrimidine-(1H, 3H)2,4-dione.

Heterocyclyl is especially a five, six or seven-membered heterocyclic system with one or two heteroatoms selected from the group comprising nitrogen, oxygen, and sulfur, which may be unsaturated or wholly or partly saturated, and is unsubstituted or substituted especially by lower alkyl, such as methyl, phenyl-lower alkyl, such as benzyl, oxo, or heteroaryl, such as 2-piperazinyl; heterocyclyl is especially 2- or 3-pyrrolidinyl, 2-oxo-5-pyrrolidinyl, piperidinyl, N-benzyl-4-piperidinyl, N-lower alkyl-4-piperidinyl, N-lower alkyl-piperazinyl, morpholinyl, e.g., 2- or 3-morpholinyl, 2-oxo-1H-azepin-3-yl, 2-tetrahydrofuranyl, or 2-methyl-1,3-dioxolan-2-yl.

Salts are especially the pharmaceutically acceptable salts of compounds of formula (I).

Such salts are formed, e.g., as acid addition salts, preferably with organic or inorganic acids, from compounds of formula (I) with a basic nitrogen atom, especially the pharmaceutically acceptable salts. Suitable inorganic acids are, e.g., halogen acids, such as hydrochloric acid, sulfuric acid, or phosphoric acid. Suitable organic acids are, e.g., carboxylic, phosphonic, sulfonic or sulfamic acids, e.g., acetic acid, propionic acid, octanoic acid, decanoic acid, dodecanoic acid, glycolic acid, lactic acid, fumaric acid, succinic acid, adipic acid, pimelic acid, suberic acid, azelaic acid, malic acid, tartaric acid, citric acid, amino acids, such as glutamic acid or aspartic acid, maleic acid, hydroxymaleic acid, methylmaleic acid, cyclohexanecarboxylic acid, adamantanecarboxylic acid, benzoic acid, salicylic acid, 4-aminosalicylic acid, phthalic acid, phenylacetic acid, mandelic acid, cinnamic acid, methane- or ethane-sulfonic acid, 2-hydroxyethanesulfonic acid, ethane-1,2-disulfonic acid, benzenesulfonic acid, 2-naphthalenesulfonic acid, 1,5-naphthalene-disulfonic acid, 2-, 3- or 4-methylbenzenesulfonic acid, methylsulfuric acid, ethylsulfuric acid, dodecylsulfuric acid, N-cyclohexylsulfamic acid, N-methyl-, N-ethyl- or N-propyl-sulfamic acid, or other organic protonic acids, such as ascorbic acid.

In the presence of negatively charged radicals, such as carboxy or sulfo, salts may also be formed with bases, e.g., metal or ammonium salts, such as alkali metal or alkaline earth metal salts, e.g., sodium, potassium, magnesium or calcium salts, or ammonium salts with ammonia or suitable organic amines, such as tertiary monoamines, e.g., triethylamine or tri(2-hydroxyethyl)amine, or heterocyclic bases, e.g., N-ethyl-piperidine or N,N′-dimethylpiperazine.

When a basic group and an acid group are present in the same molecule, a compound of formula (I) may also form internal salts.

For isolation or purification purposes it is also possible to use pharmaceutically unacceptable salts, e.g., picrates or perchlorates. For therapeutic use, only pharmaceutically acceptable salts or free compounds are employed (where applicable in the form of pharmaceutical preparations), and these are therefore preferred.

In view of the close relationship between the novel compounds in free form and those in the form of their salts, including those salts that can be used as intermediates, e.g., in the purification or identification of the novel compounds, any reference to the free compounds hereinbefore and hereinafter is to be understood as referring also to the corresponding salts, as appropriate and expedient.

Compounds within the scope of formula (I) and the process for their manufacture are disclosed in WO 04/005281 published on Jan. 15, 2004 which is hereby incorporated into the present application by reference. A preferred compound is 4-methyl-3-[[4-(3-pyridinyl)-2-pyrimidinyl]amino]-N-[5-(4-methyl-1H-imidazol-1-yl)-3-(trifluoromethyl)phenyl]benzamide.

Flt-3 inhibitors are, e.g., compounds having an IC₅₀ value in the range of 1-10,000 nM, preferably in the range of 1-100 nM in the following assays:

Flt3 kinase inhibition is determined as follows: The baculovirus donor vector pFbacG01 (GIBCO) is used to generate a recombinant baculovirus expressing the amino acid region amino acids 563-993 of the cytoplasmic kinase domain of human Flt-3. The coding sequence for the cytoplasmic domain of Flt-3 is amplified by PCR from human c-DNA libraries (Clontech). The amplified DNA fragments and the pFbacG01 vector are made compatible for ligation by digestion with BamH1 and HindIII. Ligation of these DNA fragments results in the baculovirus donor plasmid Flt-3(1.1). The production of the viruses, the expression of proteins in Sf9 cells and the purification of the GST-fused proteins are performed as follows:

Production of virus: Transfer vector (pFbacG01-Flt-3) containing the Flt-3 kinase domain is transfected into the DH10Bac cell line (GIBCO) and the transfected cells are plated on selective agar plates. Colonies without insertion of the fusion sequence into the viral genome (carried by the bacteria) are blue. Single white colonies are picked and viral DNA (bacmid) is isolated from the bacteria by standard plasmid purification procedures. Sf9 or Sf21 cells (American Type Culture Collection) are then transfected in flasks with the viral DNA using Cellfectin reagent.

Determination of small scale protein expression in Sf9 cells: Virus containing media is collected from the transfected cell culture and used for infection to increase its titre. Virus containing media obtained after two rounds of infection is used for large-scale protein expression. For large-scale protein expression 100 cm² round tissue culture plates are seeded with 5×10⁷ cells/plate and infected with 1 mL of virus-containing media (approximately 5 MOIs). After 3 days, the cells are scraped off the plate and centrifuged at 500 rpm for 5 minutes. Cell pellets from 10-20, 100 cm² plates, are resuspended in 50 mL of ice-cold lysis buffer (25 mMTris-HCl, pH 7.5, 2 mM EDTA, 1% NP-40, 1 mM DTT, 1 mM PMSF). The cells are stirred on ice for 15 minutes and then centrifuged at 5,000 rpms for 20 minutes.

Purification of GST-tagged proteins: The centrifuged cell lysate is loaded onto a 2 mL glutathione-sepharose column (Pharmacia) and washed three times with 10 mL of 25 mM Tris-HCl, pH 7.5, 2 mM EDTA, 1 mM DTT, 200 mM NaCl. The GST-tagged protein is then eluted by 10 applications (1 mL each) of 25 mM Tris-HCl, pH 7.5, 10 mM reduced-glutathione, 100 mM NaCl, 1 mM DTT, 10% Glycerol and stored at −70° C.

Measurement of enzyme activity: Tyrosine protein kinase assays with purified GST-Flt-3 are carried out in a final volume of 30 μL containing 200-1,800 ng of enzyme protein (depending on the specific activity), 20 mM Tris-HCl, pH 7.6, 3 mM MnCl₂, 3 mM MgCl₂, 1 mM DTT, 10 μM Na₃VO₄, 3 μg/mL poly(Glu,Tyr) 4:1, 1% DMSO, 8.0 μM ATP and 0.1 μCi [γ³³ P] ATP). The activity is assayed in the presence or absence of inhibitors, by measuring the incorporation of ³³P from [γ³³P] ATP into the poly(Glu,Tyr) substrate. The assay (30 μL) is carried out in 96-well plates at ambient temperature for 20 minutes under conditions described below and terminated by the addition of 20 μL of 125 mM EDTA. Subsequently, 40 μL of the reaction mixture is transferred onto Immobilon-PVDF membrane (Millipore, Bedford, Mass., USA) previously soaked for 5 minutes with methanol, rinsed with water, then soaked for 5 minutes with 0.5% H₃PO₄ and mounted on vacuum manifold with disconnected vacuum source. After spotting all samples, vacuum is connected and each well rinsed with 200 μL 0.5% H₃PO₄. Membranes are removed and washed 4× on a shaker with 1.0% H₃PO₄, once with ethanol. Membranes are counted after drying at ambient temperature, mounting in Packard TopCount 96-well frame, and addition of 10 μL/well of Microscint™ (Packard). IC₅₀ values are calculated by linear regression analysis of the percentage inhibition of each compound in duplicate, at four concentrations (usually 0.01, 0.1, 1 and 10 μM). One unit of protein kinase activity is defined as 1 nmole of ³³P ATP transferred from [γ³³P] ATP to the substrate protein per minute per mg of protein at 37° C. The compounds of the formula (I) here shown IC₅₀ values in the range between 0.005 and 20 μM, preferably between 0.01 and 10 μM.

Suitable Flt-3 inhibitors include, e.g.,

A. Compounds as disclosed in WO 03/037347, e.g staurosporine derivatives of formula (IV) or (V):

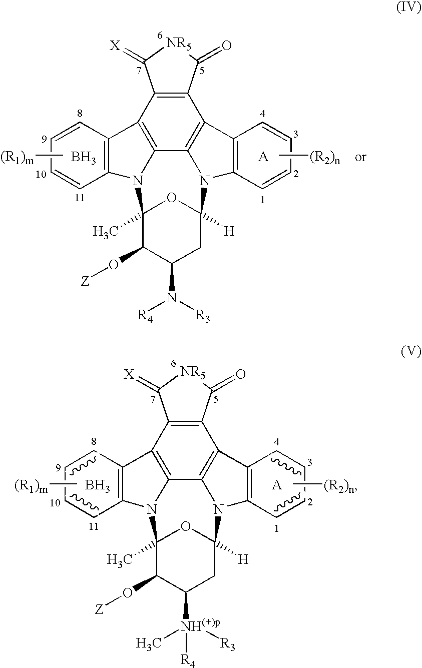

wherein (V) is the partially hydrogenated derivative of compound (IV), or

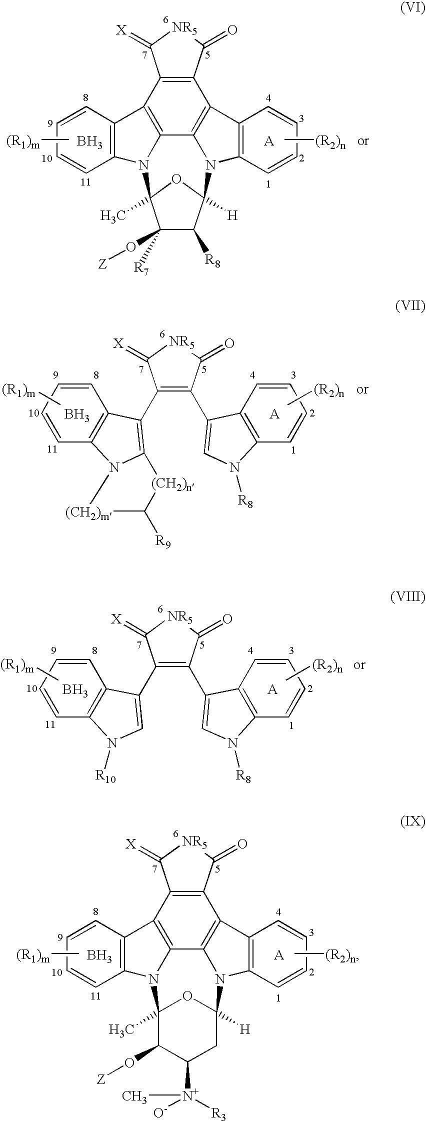

wherein
R₁ and R₂, are, independently of one another, unsubstituted or substituted alkyl, hydrogen, halogen, hydroxy, etherified or esterified hydroxy, amino, mono- or disubstituted amino, cyano, nitro, mercapto, substituted mercapto, carboxy, esterified carboxy, carbamoyl, N-mono- or N,N-di-substituted carbamoyl, sulfo, substituted sulfonyl, aminosulfonyl or N-mono- or N,N-di-substituted aminosulfonyl;
n and m are, independently of one another, a number from and including 0 to and including 4;
n′ and m′ are, independently of one another, a number from and including 0 to and including 4;
R₃, R₄, R₈ and R₁₀ are, independently of one another, hydrogen, —O⁻, acyl with up to 30 carbon atoms, an aliphatic, carbocyclic, or carbocyclic-aliphatic radical with up to 29 carbon atoms in each case, a heterocyclic or heterocyclic-aliphatic radical with up to 20 carbon atoms in each case, and in each case up to 9 heteroatoms, an acyl with up to 30 carbon atoms, wherein R₄ may also be absent, or
if R₃ is acyl with up to 30 carbon atoms, R₄ is not an acyl;
p is 0 if R₄ is absent, or is 1 if R₃ and R₄ are both present and in each case are one of the aforementioned radicals;
R₅ is hydrogen, an aliphatic, carbocyclic, or carbocyclic-aliphatic radical with up to 29 carbon atoms in each case, or a heterocyclic or heterocyclic-aliphatic radical with up to 20 carbon atoms in each case, and in each case up to 9 heteroatoms, or acyl with up to 30 carbon atoms;
R₇, R₆ and R₉ are acyl or -(lower alkyl)-acyl, unsubstituted or substituted alkyl, hydrogen, halogen, hydroxy, etherified or esterified hydroxy, amino, mono- or disubstituted amino, cyano, nitro, mercapto, substituted mercapto, carboxy, carbonyl, carbonyldioxy, esterified carboxy, carbamoyl, N-mono- or N,N-di-substituted carbamoyl, sulfo, substituted sulfonyl, aminosulfonyl or N-mono- or N,N-di-substituted aminosulfonyl;
X stands for 2 hydrogen atoms; for 1 hydrogen atom and hydroxy; for 0; or for hydrogen and lower alkoxy;
Z stands for hydrogen or lower alkyl;
and either the two bonds characterized by wavy lines are absent in ring A and replaced by 4 hydrogen atoms, and the two wavy lines in ring B each, together with the respective parallel bond, signify a double bond, or
the two bonds characterized by wavy lines are absent in ring B and replaced by a total of 4 hydrogen atoms, and the two wavy lines in ring A each, together with the respective parallel bond, signify a double bond, or
both in ring A and in ring B all of the 4 wavy bonds are absent and are replaced by a total of 8 hydrogen atoms;
   or a salt thereof, if at least one salt-forming group is present.

Preferably the FLT-3 inhibitor is N-[(9S,10R,11R,13R)-2,3,10,11,12,13-hexahydro-10-methoxy-9-methyl-1-oxo-9,13-epoxy-1H,9H-diindolo[1,2,3-gh:3′,2′,1′-Im]pyrrolo[3,4-j][1,7]benzodiazonin-11-yl]-N-methylbenzamide of the formula (X):

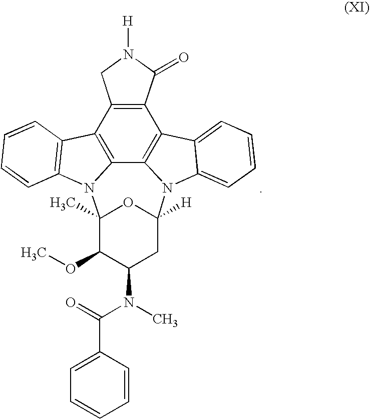

B. Compounds as disclosed in WO 03/099771, e.g., diaryl urea derivatives of the formula (XI):

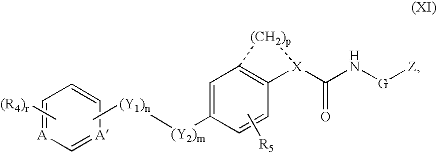

wherein
G is either not present, lower alkylene or C₃-C₅cycloalkylene; and
Z is a radical of the formula (XIa):

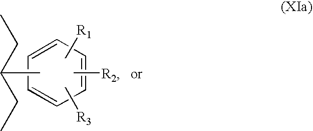

G is not present; and
Z is a radical of the formula (XIb):

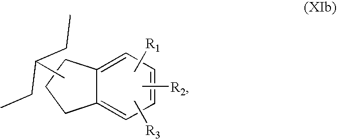

A is CH, N or N→O and A′ is N or N→O, with the proviso that not more than one of A and A′ can be N→O;
n is 1 or 2;
m is 0, 1 or 2;
p is O, 2 or 3;
r is 0 to 5;
X is NR if p is 0, wherein R is hydrogen or an organic moiety, or if p is 2 or 3, X is nitrogen which together with (CH₂)ₚ and the bonds represented in dotted (interrupted) lines (including the atoms to which they are bound) forms a ring, or
X is CHK wherein K is lower alkyl or hydrogen and p is zero, with the proviso that the bonds represented in dotted lines, if p is zero, are absent;
Y₁ is O, S or CH₂;
Y₂ is O, S or NH, with the proviso that (Y₁)ₙ—(Y₂)ₘ does not include 0-O, S—S, NH—O, NH—S or S-0 groups;
each of R₁, R₂, R₃ and R₅, independently of the others, is hydrogen or an inorganic or organic moiety or any two of them together form a lower alkylene-dioxy bridge bound via the oxygen atoms, and the remaining one of these moieties is hydrogen or an inorganic or organic moiety; and
R₄ (if present, that is, if r is not zero) is an inorganic or organic moiety;
   or a tautomer thereof; or a pharmaceutically acceptable salt thereof.

Examples of compounds of formula (XI) include:
N-(4-Pyridin-4-yl-oxy-phenyl)-N′-(4-ethyl-phenyl)-urea;
N-(4-Pyridin-4-yl-oxy-phenyl)-N′-(3-trifluoromethyl-phenyl)-urea;
N-(4-Pyridin-4-yl-oxy-phenyl)-N′-(4-(2,2,2-trifluoroethoxy)-3-trifluoromethyl-phenyl)-urea;
N-(4-(4-(4-Hydroxyphenylamino)-pyrimidin-6-yl)-oxyphenyl)-N′-(3-trifluoromethylphenyl)-urea;
N-(4-(2-Methyl-pyridin-4-yl)-oxyphenyl)-N′-(3-trifluoromethyl-phenyl)-urea;
N-(4-Pyridin-4-yl-oxy-phenyl)-N′-(4-n-propyl-phenyl)-urea;
N-(4-Pyridin-4-yl-oxy-phenyl)-N′-(4-methyl-phenyl)-urea;
N-Methyl-N-(4-pyridin-4-yl-oxy-phenyl)-N′-(4-ethyl-phenyl)-urea;
N-Methyl-N-(4-pyridin-4-yl-oxy-phenyl)-N′-(3-trifluoromethyl-phenyl)-urea;
N-Methyl-N-(4-pyridin-4-yl-oxy-phenyl)-N′-(4-n-propyl-phenyl)-urea;
N-Methyl-N-(4-pyridin-4-yl-oxy-phenyl)-N′-(4-methyl-phenyl)-urea;
N-(4-Pyridin-4-yl-oxy-phenyl)-N′-(4-bromo-3-trifluoromethyl-phenyl)-urea;
N-(4-Pyridin-4-yl-oxy-phenyl)-N′-(3-methoxy-5-trifluoromethyl-phenyl)-urea;
N-(4-Pyridin-4-ylmethyl-phenyl)-N′-(4-n-propyl-phenyl)-urea;
N-(4-Pyridin-4-ylmethyl-phenyl)-N′-(4-ethyl-phenyl)-urea;
N-(4-Pyridin-4-ylmethyl-phenyl)-N′-(4-methyl-phenyl)-urea;
N-(4-Pyridin-4-ylmethyl-phenyl)-N′-(3-trifluoromethyl-phenyl)-urea;
N-(4-Pyridin-4-yl-oxy-phenyl)acetyl-(4-ethyl-phenyl)-amide;
N-(4-Pyridin-4-yl-oxy-phenyl)acetyl-(4-methyl-phenyl)-amide;
N-(4-Pyridin-4-yl-oxy-phenyl)acetyl-(4-n-propyl-phenyl)-amide;
5-(4-Pyridyl-oxy)-N-(3-trifluoromethyl-phenyl)amino-carbonyl-2,3-dihydroindole;
5-(4-Pyridyl-oxy)-N-(3-trifluoromethyl-phenyl)amino-carbonyl-1,2,3,4-tetrahydroquinoline;
N-(4-(4-Chloropyrimidin-6-yl)-oxyphenyl)-N′-(3-trifluoromethylphenyl)-urea;
N-(4-Pyridin-4-yl-oxyphenyl)-N′-(4-phenyl-3-trifluoromethyl-phenyl)-urea;
N-(4-Pyridin-4-yl-oxyphenyl)-N′-(4-(piperidin-1-yl)-3-trifluoromethyl-phenyl)-urea;
N-(4-Pyridin-4-yl-oxyphenyl)-N′-(4-(morpholino)-3-trifluoromethyl-phenyl)-urea;
N-(4-Pyridin-4-yl-oxyphenyl)-N′-(3,4,5-trimethoxy-phenyl)-urea;
N-(4-Pyridin-4-yl-oxyphenyl)-N′-(3-methoxy-4-phenyl-phenyl)-urea;
N-(4-Pyridin-4-yl-oxyphenyl)-N′-(3-methoxy-4,5-(ethylen-1,2-dioxy)-phenyl)-urea;
N-(4-Pyridin-4-yl-oxyphenyl)-N′-(3-methoxy-4-(2,2,2-trifluoroethoxy)-phenyl)-urea;
N-(4-Pyridin-4-yl-oxyphenyl)-N′-(3-methoxy-4-piperidin-1-yl-phenyl)-urea;
N-(4-Pyridin-4-yl-oxyphenyl)-N′-(4-piperidin-1-yl-phenyl)-urea;
N-(4-[2-(4-Hydroxyphenyl)-amino-pyrimidin-4-yl]-oxyphenyl-N′-(3-trifluoromethyl-phenyl)-urea;
N-(4-[4-(4-Sulfamoylphenyl)-amino-pyrimidin-6-yl]-oxyphenyl-N′-(3-trifluoromethyl-phenyl)-urea;
N-(4-[4-(4-Carbamoylphenyl)-amino-pyrimidin-6-yl]-oxyphenyl-N′-(3-trifluoromethyl-phenyl)-urea;
N-(4-[4-(4-(N-2-Hydroxyethylcarbamoyl)-phenyl)-amino-pyrimidin-6-yl]-oxyphenyl-N′-(3-trifluoromethyl-phenyl)-urea;
N-(4-[4-(4-Hydroxyphenyl)-amino-pyrimidin-6-yl]-oxyphenyl-N′-(3-trifluoromethyl-4-(2,2,2-trifluoroethoxy)-phenyl)-urea;
N-(4-(N-Oxido-pyridin-4-yl)-oxyphenyl)-N′-(3-trifluoromthyl-phenyl)-urea;
N-(4-(2-Methoxypyridin-5-yl)-oxyphenyl)-N′-(3-trifluoromethyl-phenyl)-urea;
N-(4-(2-Pyridon-5-yl)-oxyphenyl)-N′-(3-trifluoromethyl-phenyl)-urea;
N-[4-{(2-Acetylamino)-pyridin-4-yl}-oxy]-phenyl-N′-(3-trifluoromethyl-phenyl)-urea;
N-[4-(Pyridin-4-yl-oxy)-2-chloro-phenyl]-N′-(3-trifluoromethyl-phenyl)-urea;
N-[4-(Pyridin-4-yl-oxy)-2-methyl-phenyl]-N′-(3-trifluoromethyl-phenyl)-urea; and
N-(4-[4-(2-Aminoethoxyphenyl)-amino-pyrimidin-6-yl]-oxyphenyl-N′-(3-trifluoromethyl-phenyl)-urea,
   or pharmaceutically acceptable salts thereof. Most preferred is 1-[4-(4-ethyl-piperazinyl-1-ylmethyl)-3-trifluoromethyl-phenyl]-3-[4-(6-methylamino-pyrimidin-4-yloxy-phenyl]-urea; 1-[4-(2-amino-pyrimidin-4-yloxy)-phenyl]-3-[4-(4-ethyl-piperazin-1-ylmethyl)-3-trifluoromethyl-phenyl]-urea and 1-[4-(2-amino-pyrimidin-4-yloxy)-phenyl]-3-[4-(4-methyl-piperazin-1-ylmethyl)-3-trifluoromethyl-phenyl]-urea.
   C. Compounds as disclosed in WO 04/046120, e.g., compounds of the formula (XII):

A compound of formula (XI):

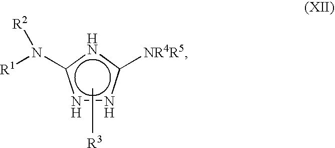

or a pharmaceutically acceptable salt thereof,
wherein
R₁ is hydrogen or Y—R′, wherein Y is an optionally substituted C₁-C₆alkylidene chain wherein up to two methylene units are optionally and independently replaced with —O—, —S—, —NR—, —OCO—, —COO— or —CO—; each occurrence of R is independently hydrogen or an optionally substituted C₁-C₆aliphatic group; and each occurrence of R′ is independently hydrogen or an optionally substituted group selected from a C₁-C₆aliphatic group, a 3- to 8-membered saturated, partially unsaturated or fully unsaturated monocyclic ring having 0-3 heteroatoms independently selected from nitrogen, oxygen or sulfur, or an 8- to 12-membered saturated, partially unsaturated or fully unsaturated bicyclic ring system having 0-5 heteroatoms independently selected from nitrogen, oxygen or sulfur; or R and R′, two occurrences of R, or two occurrences of R′, are taken together with the atom(s) to which they are bound to form an optionally substituted 3- to 12-membered saturated, partially unsaturated or fully unsaturated monocyclic or bicyclic ring having 0-4 heteroatoms independently selected from nitrogen, oxygen or sulfur;
R² is -(T)ₙAr¹ or -(T)ₙCy¹, wherein T is an optionally substituted C₁-C₄alkylidene chain wherein one methylene unit of T is optionally replaced by —NR—, —S—, —O—, —CS—, —CO₂—, —OCO—, —CO—, —COCO—, —CONR—, —NRCO—, —NRCO₂—, —SO₂NR—, —NRSO₂—, —CONRNR—, —NRCONR—, —OCONR—, —NRNR—, —NRSO₂NR—, —SO—, —SO₂—, —PO—, —PO₂— or —POR—;
   n is 0 or 1;
   Ar¹ is an optionally substituted aryl group selected from a 5- to 6-membered monocyclic or an 8- to 12-membered bicyclic ring having 0-5 heteroatoms independently selected from nitrogen, oxygen or sulfur; and
   Cy¹ is an optionally substituted group selected from a 3- to 7-membered saturated or partially unsaturated monocyclic ring having 0-3 heteroatoms independently selected from nitrogen, oxygen or sulfur, or an 8- to 12-membered saturated or partially unsaturated bicyclic ring system having 0-5 heteroatoms independently selected from nitrogen, oxygen or sulfur, or
R¹ and R², taken together with the nitrogen form an optionally substituted 5- to 8-membered monocyclic or 8- to 12-membered bicyclic saturated, partially unsaturated or fully unsaturated ring having 0-3 additional heteroatoms independently selected from nitrogen, oxygen or sulfur, wherein Ar¹, Cy¹ or any ring formed by R¹ and R² taken together, are each independently optionally substituted with x independent occurrences of Q-R^{x}, wherein x is 0-5, Q is a bond or is a C₁-C₆alkylidene chain wherein up to two methylene units of Q are optionally replaced by —NR—, —S—, —O—, —CS—, —CO₂—, —OCO—, —CO—, —COCO—, —CONR—, —NRCO—, —NRCO₂—, —SO₂NR—, —NRSO₂—, —CONRNR—, —NRCONR—, —OCONR—, —NRNR—, —NRSO₂NR—, —SO—, —SO₂—, —PO—, —PO₂— or —POR—; and each occurrence of R^{x} is independently R′, halogen, NO₂, CN, OR′, SR′, N(R′)₂, NR′COR′, NR′CONR′₂, NR′CO₂R′, COR′, CO₂R′, OCOR′, CON(R′)₂, OCON(R′)₂, SOR′, SO₂R′, SO₂N(R′)₂, NR′SO₂R′, NR′SO₂N(R′)₂, COCOR′ or COCH₂COR′;
R³ is bonded to the nitrogen atom in either the 1- or 2-position of the ring and is (L)ₘAr² or (L)ₘCy², wherein L is an optionally substituted C₁-C₄alkylidene chain wherein one methylene unit of L is optionally replaced by —NR—, —S—, —O—, —CS—, —CO₂—, —OCO—, —CO—, —COCO—, —CONR—, —NRCO—, —NRCO₂—, —SO₂NR—, —NRSO₂—, —CONRNR—, —NRCONR—, —OCONR—, —NRNR—, —NRSO₂NR—, —SO—, —SO₂—, —PO—, —PO₂— or —POR—;
   m is 0 or 1;
   Ar² is an optionally substituted aryl group selected from a 5- to 6-membered monocyclic or an 8- to 12-membered bicyclic ring having 0-5 heteroatoms independently selected from nitrogen, oxygen or sulfur; and
   Cy² is an optionally substituted group selected from a 3- to 7-membered saturated or partially unsaturated monocyclic ring having 0-3 heteroatoms independently selected from nitrogen, oxygen or sulfur, or an 8- to 12-membered saturated or partially unsaturated bicyclic ring system having 0-5 heteroatoms independently selected from nitrogen, oxygen or sulfur, wherein Ar² and Cy² are each independently optionally substituted with y occurrences of Z-R^{Y}; wherein y is 0-5, Z is a bond or is a C₁-C₆alkylidene chain wherein up to two methylene units of Z are optionally replaced by —NR—, —S—, —O—, —CS—, —CO₂—, —OCO—, —CO—, —COCO—, —CONR—, —NRCO—, —NRCO₂—, —SO₂NR—, —NRSO₂—, —CONRNR—, —NRCONR—, —OCONR—, —NRNR—, —NRSO₂NR—, —SO—, —SO₂—, —PO—, —PO₂— or —POR—; and each occurrence of R^{Y} is independently R′, halogen, NO₂, CN, OR′, SR′, N(R′)₂, NR′COR′, NR′CONR′₂, NR′CO₂R′, COR′, CO₂R′, OCOR′, CON(R′)₂, OCON(R′)₂, SOR′, SO₂R′, SO₂N(R′)₂, NR′SO₂R′, NR′SO₂N(R′)₂, COCOR′ or COCH₂COR′;
R⁴ is hydrogen or C₁-C₆alkyl, provided that when R⁵ is hydrogen, R⁴ is also hydrogen;
R⁵ is hydrogen, or
R³ and R⁵, taken together form an optionally substituted group selected from a 5- to 7-membered saturated, partially unsaturated or fully unsaturated monocyclic ring having 0-3 heteroatoms independently selected from nitrogen, oxygen or sulfur, or an 8- to 10-membered saturated, partially unsaturated or fully unsaturated bicyclic ring system having 0-3 heteroatoms independently selected from nitrogen, oxygen or sulfur; and
wherein any ring formed R³ and R⁵ taken together, is optionally substituted with up to five substituents selected from W—R^{W}, wherein W is a bond or is a C₁-C₆alkylidene chain wherein up to two methylene units of W are optionally and independently replaced by —NR—, —S—, —O—, —CS—, —CO₂—, —OCO—, —CO—, —COCO—, —CONR—, —NRCO—, —NRCO₂—, —SO₂NR—, —NRSO₂—, —CONRNR—, —NRCONR—, —OCONR—, —NRNR—, —NRSO₂NR—, —SO—, —SO₂—, —PO—, —PO₂— or —POR—; and each occurrence of R^{W} is independently R′, halogen, NO₂, CN, OR′, SR′, N(R′)₂, NR′COR′, NR′CONR′₂, NR′CO₂R′, COR′, CO₂R′, OCOR′, CON(R′)₂, OCON(R′)₂, SOR′, SO₂R′, SO₂N(R′)₂, NR′SO₂R′, NR′SO₂N(R′)₂, COCOR′ or COCH₂COR′,
   provided that when:
a) R³ is unsubstituted phenyl and R¹ is hydrogen, then R² is not:
   i) unsubstituted phenyl;
   ii) unsubstituted pyridyl;
   iii) benzyl substituted with o-OMe;
   iv) —(C═S)NH(C═O)phenyl;
   v)

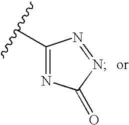

vi) —(C═S)NH-naphthyl or —(C═O)NH-naphthyl; or
b) R³ is substituted or unsubstituted phenyl, then R² is not phenyl substituted in the para position with oxazole, thiazole, thiadiazole, oxadiazole, tetrazole, triazole, diazole or pyrrole;
c) R³ is phenyl, pyridyl, pyrimidinedione or cyclohexyl, and R¹ is hydrogen, then R² is not phenyl simultaneously substituted with one occurrence of OMe in the meta position, and one occurrence of oxazole in the para position;
d) R³ is 4-Cl phenyl or 3,4-Cl-phenyl, then R² is not p-Cl phenyl;
e) R³ is unsubstituted pyrimidinyl, then R² is not unsubstituted phenyl, p-OMe substituted phenyl, p-OEt substituted phenyl or o-OMe substituted phenyl or when R³ is 4-Me pyrimidinyl or 4,6-dimethylpyrimidinyl, then R² is not unsubstituted phenyl;
f) compounds of formula (XII) exclude:

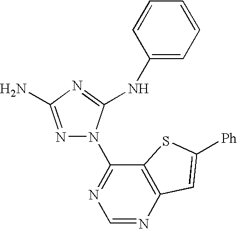

g) R² is 3-pyridinyl and R¹ is hydrogen, then R³ is not trimethoxybenzoyl;
h) R³ is optionally substituted phenyl and R¹ is hydrogen, then R² is not —(C═S)NH(C═O)phenyl, —(C═O)NHphenyl, —(C═S)NHphenyl or —(C═O)CH₂(C═O)-phenyl;
i) R¹ is hydrogen and R² is unsubstituted benzyl, then R³ is not thiadiazole substituted with optionally substituted phenyl;
j) R¹ is hydrogen, R² is pyridyl and R³ is pyridyl, then R² is not substituted with one or more of CF₃, Me, OMe, Br or Cl;
k) R¹ is hydrogen and R² is pyridyl, then R³ is not unsubstituted pyridyl, unsubstituted quinoline, unsubstituted phenyl or unsubstituted isoquinoline;
l) R¹ is hydrogen and R² is unsubstituted quinoline, then R³ is not unsubstituted pyridyl or unsubstituted quinoline;
m) R¹ is hydrogen and R² is unsubstituted isoquinoline or unsubstituted naphthyl, then R³ is not unsubstituted pyridyl;
n) compounds of formula (XII) exclude those compounds having the general structure:

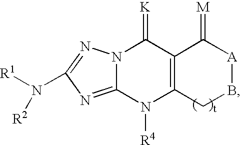

wherein
   R¹, R² and R³ are as defined above;
   M and K are O or H₂, provided that K and M are different, A and B are each —CH₂—, —NH—, —N-alkyl-, N-aralkyl-, —NCOR^{a}, —NCONHR^{b} or —NCSNHR^{b}, wherein R^{a} is lower alkyl or aralkyl; and R^{b} is straight- or branched-chain alkyl, aralkyl or aryl which can either be unsubstituted or substituted with one or more alkyl and/or haloalkyl substituents;
o) compounds of formula (XII) exclude those compounds having the general structure:

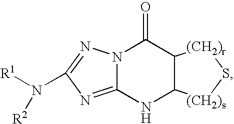

wherein
   R¹ and R² are as defined above; and
   r and s are each independently 0, 1, 2, 3 or 4, provided that the sum of s and r is at least 1;
p) compounds of formula (XII) exclude any one or more of, or all of the following compounds:

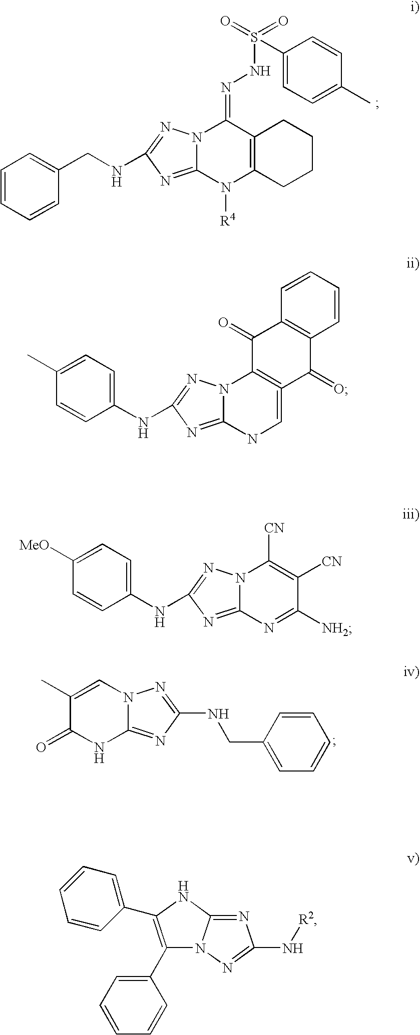

where R² is NH(CH)(Ph)C═O(Ph);

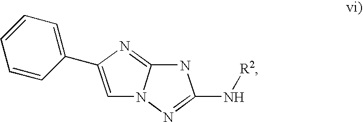

where R² is unsubstituted phenyl or phenyl substituted with OMe, Cl or Me;

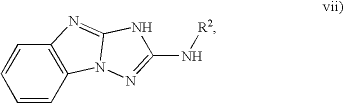

where
   R² is unsubstituted phenyl or phenyl substituted with OMe, Cl, Me or OMe, or
   R² is unsubstituted benzyl;

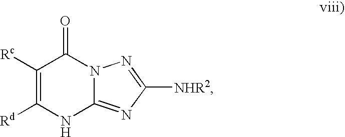

where
   R² is optionally substituted aralkyl; and
   R^{c} and R^{d} are, each independently, Me, hydrogen, CH₂Cl or Cl;

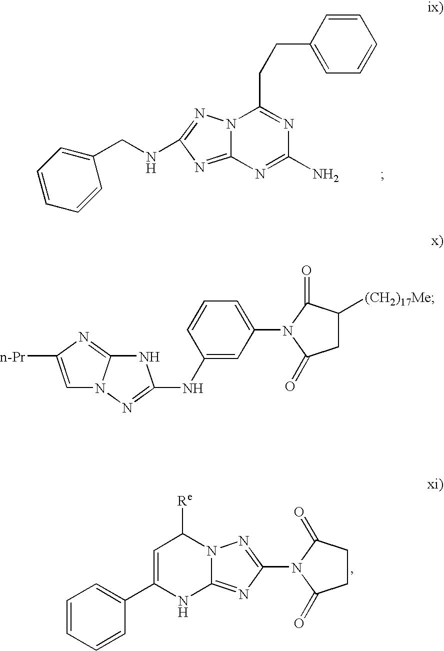

where R^{e} is optionally substituted phenyl;

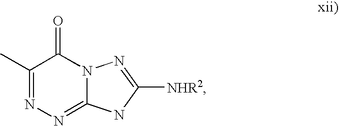

where R² is phenyl optionally substituted with Me, OMe, Br or Cl; or
q) when
   R¹ is hydrogen; and
   R² is phenyl or optionally substituted phenyl; and
   m is 1, then L is not —CO—, —COCH₂— or —COCH═CH—.

In each case where citations of patent applications are given above, the subject matter relating to the compounds is hereby incorporated into the present application by reference. Comprised are likewise the pharmaceutically acceptable salts thereof, the corresponding racemates, diastereoisomers, enantiomers, tautomers, as well as the corresponding crystal modifications of above disclosed compounds where present, e.g. solvates, hydrates and polymorphs, which are disclosed therein. The compounds used as active ingredients in the combinations of the invention can be prepared and administered as described in the cited documents, respectively. Also within the scope of this invention is the combination of more than two separate active ingredients as set forth above, i.e., a pharmaceutical combination within the scope of this invention could include three active ingredients or more.

In accordance with the particular findings of the present invention, there is provided
1. A pharmaceutical combination comprising:
   a) a pyrimidylaminobenzamide compound of formula (I); and
   b) at least one Flt-3 inhibitor.
2. A method for treating or preventing proliferative disease in a subject in need thereof, comprising co-administration to said subject, e.g., concomitantly or in sequence, of a therapeutically effective amount of a pyrimidylaminobenzamide compound of formula (I) and a Flt-3 inhibitor, e.g., as disclosed above.
Examples of proliferative diseases include, e.g., tumors, leukemias, psoriasis, restenosis, sclerodermitis and fibrosis.
3. A pharmaceutical combination as defined under 1) above, e.g. for use in a method as defined under 2) above.
4. A pharmaceutical combination as defined under 1) above for use in the preparation of a medicament for use in a method as defined under 2) above.

Utility of the combination of the invention in a method as hereinabove specified, may be demonstrated in animal test methods as well as in clinic, e.g., in accordance with the methods hereinafter described.

It has now surprisingly been found that the combination of a pyrimidylaminobenzamide compound and a Flt-3 inhibitor possesses therapeutic properties, which render it particularly useful as a treatment for proliferative diseases.

In another embodiment, the instant invention provides a method for treating proliferative diseases comprising administering to a mammal in need of such treatment a therapeutically effective amount of the combination of a pyrimidylaminobenzamide compound and a Flt-3 inhibitor or pharmaceutically acceptable salts or prodrugs thereof.

Preferably the instant invention provides a method for treating mammals, especially humans, suffering from proliferative diseases comprising administering to a mammal in need of such treatment an inhibiting amount of the combination of a pyrimidylaminobenzamide compound and a Flt-3 inhibitor or pharmaceutically acceptable salts thereof.

In the present description, the term “treatment” includes both prophylactic or preventative treatment, as well as curative or disease suppressive treatment, including treatment of patients at risk of contracting the disease or suspected to have contracted the disease, as well as ill patients. This term further includes the treatment for the delay of progression of the disease.

The term “curative”, as used herein, means efficacy in treating ongoing episodes involving proliferative diseases.

The term “prophylactic” means the prevention of the onset or recurrence of diseases involving proliferative diseases.

The term “delay of progression”, as used herein, means administration of the active compound to patients being in a pre-stage or in an early phase of the disease to be treated, in which patients, e.g., a pre-form of the corresponding disease is diagnosed or which patients are in a condition, e.g., during a medical treatment or a condition resulting from an accident, under which it is likely that a corresponding disease will develop.

This unforeseeable range of properties means that the use of the combination of a pyrimidylaminobenzamide compound and a Flt-3 inhibitor are of particular interest for the manufacture of a medicament for the treatment of proliferative diseases.

To demonstrate that the combination of a pyrimidylaminobenzamide compound and a Flt-3 inhibitor is particularly suitable for the treatment of proliferative diseases with good therapeutic margin and other advantages, clinical trials can be carried out in a manner known to the skilled person.

### A. Combined Treatment

Suitable clinical studies are, e.g., open-label, dose escalation studies in patients with proliferative diseases. Such studies prove in particular the synergism of the active ingredients of the combination of the invention. The beneficial effects can be determined directly through the results of these studies which are known as such to a person skilled in the art. Such studies are, in particular, suitable to compare the effects of a monotherapy using the active ingredients and a combination of the invention. Preferably, the dose of agent (a) is escalated until the Maximum Tolerated Dosage is reached, and agent (b) is administered with a fixed dose. Alternatively, the agent (a) is administered in a fixed dose and the dose of agent (b) is escalated. Each patient receives doses of the agent (a) either daily or intermittent. The efficacy of the treatment can be determined in such studies, e.g., after 12, 18 or 24 weeks by evaluation of symptom scores every 6 weeks.

The administration of a pharmaceutical combination of the invention results not only in a beneficial effect, e.g., a synergistic therapeutic effect, e.g., with regard to alleviating, delaying progression of or inhibiting the symptoms, but also in further surprising beneficial effects, e.g., fewer side effects, an improved quality of life or a decreased morbidity, compared with a monotherapy applying only one of the pharmaceutically active ingredients used in the combination of the invention.

A further benefit is that lower doses of the active ingredients of the combination of the invention can be used, e.g., that the dosages need not only often be smaller but are also applied less frequently, which may diminish the incidence or severity of side effects. This is in accordance with the desires and requirements of the patients to be treated.

The term “co-administration” or “combined administration” or the like as utilized herein are meant to encompass administration of the selected therapeutic agents to a single patient, and are intended to include treatment regimens in which the agents are not necessarily administered by the same route of administration or at the same time.

It is one objective of this invention to provide a pharmaceutical composition comprising a quantity, which is jointly therapeutically effective at targeting or preventing proliferative diseases a combination of the invention. In this composition, agent (a) and agent (b) may be administered together, one after the other or separately in one combined unit dosage form or in two separate unit dosage forms. The unit dosage form may also be a fixed combination.

The pharmaceutical compositions for separate administration of agent (a) and agent (b) or for the administration in a fixed combination, i.e., a single galenical composition comprising at least two combination partners (a) and (b), according to the invention may be prepared in a manner known per se and are those suitable for enteral, such as oral or rectal, and parenteral administration to mammals (warm-blooded animals), including humans, comprising a therapeutically effective amount of at least one pharmacologically active combination partner alone, e.g., as indicated above, or in combination with one or more pharmaceutically acceptable carriers or diluents, especially suitable for enteral or parenteral application.

Suitable pharmaceutical compositions contain, e.g., from about 0.1% to about 99.9%, preferably from about 1% to about 60%, of the active ingredient(s). Pharmaceutical preparations for the combination therapy for enteral or parenteral administration are, e.g., those in unit dosage forms, such as sugar-coated tablets, tablets, capsules or suppositories, or ampoules. If not indicated otherwise, these are prepared in a manner known perse, e.g., by means of conventional mixing, granulating, sugar-coating, dissolving or lyophilizing processes. It will be appreciated that the unit content of a combination partner contained in an individual dose of each dosage form need not in itself constitute an effective amount since the necessary effective amount can be reached by administration of a plurality of dosage units.

In particular, a therapeutically effective amount of each of the combination partner of the combination of the invention may be administered simultaneously or sequentially and in any order, and the components may be administered separately or as a fixed combination. For example, the method of preventing or treating proliferative diseases according to the invention may comprise: (i) administration of the first agent (a) in free or pharmaceutically acceptable salt form; and (ii) administration of an agent (b) in free or pharmaceutically acceptable salt form, simultaneously or sequentially in any order, in jointly therapeutically effective amounts, preferably in synergistically effective amounts, e.g., in daily or intermittently dosages corresponding to the amounts described herein. The individual combination partners of the combination of the invention may be administered separately at different times during the course of therapy or concurrently in divided or single combination forms. Furthermore, the term administering also encompasses the use of a pro-drug of a combination partner that convert in vivo to the combination partner as such. The instant invention is therefore to be understood as embracing all such regimens of simultaneous or alternating treatment and the term “administering” is to be interpreted accordingly.

The effective dosage of each of the combination partners employed in the combination of the invention may vary depending on the particular compound or pharmaceutical composition employed, the mode of administration, the condition being treated, the severity of the condition being treated. Thus, the dosage regimen of the combination of the invention is selected in accordance with a variety of factors including the route of administration and the renal and hepatic function of the patient. A clinician or physician of ordinary skill can readily determine and prescribe the effective amount of the single active ingredients required to alleviate, counter or arrest the progress of the condition. Optimal precision in achieving concentration of the active ingredients within the range that yields efficacy without toxicity requires a regimen based on the kinetics of the active ingredients' availability to target sites.

Daily dosages for agent (a) or (b) or will, of course, vary depending on a variety of factors, e.g., the compound chosen, the particular condition to be treated and the desired effect. In general, however, satisfactory results are achieved on administration of agent (a) at daily dosage rates of the order of ca. 0.03 to 5 mg/kg per day, particularly 0.1 to 5 mg/kg per day, e.g. 0.1 to 2.5 mg/kg per day, as a single dose or in divided doses. Agent (a) and agent (b) may be administered by any conventional route, in particular enterally, e.g., orally, e.g., in the form of tablets, capsules, drink solutions or parenterally, e.g., in the form of injectable solutions or suspensions. Suitable unit dosage forms for oral administration comprise from ca. 0.02 to 50 mg active ingredient, usually 0.1 to 30 mg, e.g. agent (a) or (b), together with one or more pharmaceutically acceptable diluents or carriers therefore.

Agent (b) may be administered to a human in a daily dosage range of 0.5 to 1000 mg. Suitable unit dosage forms for oral administration comprise from ca. 0.1 to 500 mg active ingredient, together with one or more pharmaceutically acceptable diluents or carriers therefore.

The administration of a pharmaceutical combination of the invention results not only in a beneficial effect, e.g., a synergistic therapeutic effect, e.g., with regard to inhibiting the unregulated proliferation of hematological stem cells or slowing down the progression of leukemias, such as CML (chronic myeloid leukemia) or AML (acute myeloid leukemia), or the growth of tumors, but also in further surprising beneficial effects, e.g., less side effects, an improved quality of life or a decreased morbidity, compared to a monotherapy applying only one of the pharmaceutically active ingredients used in the combination of the invention.

A further benefit is that lower doses of the active ingredients of the combination of the invention can be used, e.g., that the dosages need not only often be smaller but are also applied less frequently, or can be used in order to diminish the incidence of side effects. This is in accordance with the desires and requirements of the patients to be treated.

Combinations of a pyrimidylaminobenzamide compound and a Flt-3 inhibitor may be combined, independently or together, with one or more pharmaceutically acceptable carriers and, optionally, one or more other conventional pharmaceutical adjuvants and administered enterally, e.g., orally, in the form of tablets, capsules, caplets, etc. or parenterally, e.g., intraperitoneally or intravenously, in the form of sterile injectable solutions or suspensions. The enteral and parenteral compositions may be prepared by conventional means.

The combination of a pyrimidylaminobenzamide compound and a Flt-3 inhibitor can be used alone or combined with at least one other pharmaceutically active compound for use in these pathologies. These active compounds can be combined in the same pharmaceutical preparation or in the form of combined preparations “kit of parts” in the sense that the combination partners can be dosed independently or by use of different fixed combinations with distinguished amounts of the combination partners, i.e., simultaneously or at different time points. The parts of the kit of parts can then, e.g., be administered simultaneously or chronologically staggered, that is at different time points and with equal or different time intervals for any part of the kit of parts. Non-limiting examples of compounds which can be cited for use in combination with the combination of a pyrimidylaminobenzamide compound and a Flt-3 inhibitor are cytotoxic chemotherapy drugs, such as cytosine arabinoside, daunorubicin, doxorubicin, cyclophosphamide, VP-16, or imatinib etc. Further, the combination of a pyrimidylaminobenzamide compound and a Flt-3 inhibitor could be combined with other inhibitors of signal transduction or other oncogene-targeted drugs with the expectation that significant synergy would result.

### B. Diseases to be Treated

The term “proliferative disease” includes but is not restricted to tumors, psoriasis, restenosis, sclerodermitis and fibrosis.

The term “hematological malignancy” refers in particular to leukemias, especially those expressing Bcr-Abl, c-Kit or Flt-3, and includes, but is not limited to, chronic myelogenous leukemia and acute lymphocyte leukemia (ALL), especially the Philadelphia chromosome positive acute lymphocyte leukemia (Ph+ALL), as well as ST1571-resistant leukemia. Especially preferred is use of the combinations of the present invention for leukemias, such as CML or AML.

The term “a solid tumor disease” especially means ovarian cancer, breast cancer, cancer of the colon and generally the gastrointestinal tract, cervix cancer, lung cancer, e.g., small-cell lung cancer and non-small-cell lung cancer, head and neck cancer, bladder cancer, cancer of the prostate or Kaposi's sarcoma.

The combinations according to the invention, that inhibit the protein kinase activities mentioned, especially tyrosine protein kinases mentioned above and below, can therefore be used in the treatment of protein kinase dependent diseases. Protein kinase dependent diseases are especially proliferative diseases, preferably benign or especially malignant tumours (e.g., carcinoma of the kidneys, liver, adrenal glands, bladder, breast, stomach, ovaries, colon, rectum, prostate, pancreas, lungs, vagina or thyroid, sarcoma, glioblastomas and numerous tumours of the neck and head, as well as leukemias). They are able to bring about the regression of tumors and to prevent the formation of tumor metastases and the growth of (also micro)metastases. In addition they can be used in epidermal hyperproliferation (e.g., psoriasis), in prostate hyperplasia, and in the treatment of neoplasias, especially of epithelial character, for example mammary carcinoma. It is also possible to use the combinations of the present invention in the treatment of diseases of the immune system insofar as several or, especially, individual tyrosine protein kinases are involved; furthermore, the combinations of the present invention can be used also in the treatment of diseases of the central or peripheral nervous system where signal transmission by at least one tyrosine protein kinase, especially selected from those mentioned specifically, is involved.

In CML, a reciprocally balanced chromosomal translocation in hematopoietic stem cells (HSCs) produces the BCR-ABL hybrid gene. The latter encodes the oncogenic BCR-ABL fusion protein. Whereas ABL encodes a tightly regulated protein tyrosine kinase, which plays a fundamental role in regulating cell proliferation, adherence and apoptosis, the BCR-ABL fusion gene encodes as constitutively activated kinase, which transforms HSCs to produce a phenotype exhibiting deregulated clonal proliferation, reduced capacity to adhere to the bone marrow stroma and a reduces apoptotic response to mutagenic stimuli, which enable it to accumulate progressively more malignant transformations. The resulting granulocytes fail to develop into mature lymphocytes and are released into the circulation, leading to a deficiency in the mature cells and increased susceptibility to infection. ATP-competitive inhibitors of BCR-ABL have been described which prevent the kinase from activating mitogenic and anti-apoptotic pathways (e.g., P-3 kinase and STAT5), leading to the death of the BCR-ABL phenotype cells and thereby providing an effective therapy against CML. The combinations of the present invention are thus especially appropriate for the therapy of diseases related to its overexpression, especially leukemias, such as leukemias, e.g., CML or ALL.

The combinations according to the invention, that inhibit the protein kinase activities mentioned, especially tyrosine protein kinases mentioned above and below, can therefore be used in the treatment of protein kinase dependent diseases. Protein kinase dependent diseases are especially proliferative diseases, preferably benign or especially malignant tumours (e.g., carcinoma of the kidneys, liver, adrenal glands, bladder, breast, stomach, ovaries, colon, rectum, prostate, pancreas, lungs, vagina or thyroid, sarcoma, glioblastomas and numerous tumours of the neck and head, as well as leukemias). They are able to bring about the regression of tumors and to prevent the formation of tumor metastases and the growth of (also micro)metastases. In addition they can be used in epidermal hyperproliferation (e.g. psoriasis), in prostate hyperplasia, and in the treatment of neoplasias, especially of epithelial character, for example mammary carcinoma. It is also possible to use the combinations of the present invention in the treatment of diseases of the immune system insofar as several or, especially, individual tyrosine protein kinases are involved; furthermore, the combinations of the present invention can be used also in the treatment of diseases of the central or peripheral nervous system where signal transmission by at least one tyrosine protein kinase, especially selected from those mentioned specifically, is involved.

Flt3 (FMD-like tyrosine kinase) is especially expressed in hematopoietic progenitor cells and in progenitors of the lymphoid and myeloid series. Aberrant expression of the Flt3 gene has been documented in both adult and childhood leukemias including AML, AML with trilineage myelodysplasia (AML/TMDS), ALL, CML and myelodysplastic syndrome (MDS), which are therefore the preferred diseases to be treated with compounds of the formula (I). Activating mutations in Flt3 have been found in approximately 25-30% of patients with AML. Thus there is accumulating evidence for the role of Flt3 in human leukemias, and the combinations of the present invention, as Flt3 inhibitors are especially of use in the therapy of this type of diseases (see Tse et al., Leukemia 15(7), 1001-1010 (2001); Tomoki et al., Cancer Chemother. Pharmacol. 48 (Suppl. 1), S27-S30 (2001); Birkenkamp et al., Leukemia 15(12), 1923-1921 (2001); Kelly et al., Neoplasia 99(1), 310-318 (2002)).

In CML, a reciprocally balanced chromosomal translocation in HSCs produces the BCR-ABL hybrid gene. The latter encodes the oncogenic BCR-ABL fusion protein. Whereas ABL encodes a tightly regulated protein tyrosine kinase, which plays a fundamental role in regulating cell proliferation, adherence and apoptosis, the BCR-ABL fusion gene encodes as constitutively activated kinase, which transforms HSCs to produce a phenotype exhibiting deregulated clonal proliferation, reduced capacity to adhere to the bone marrow stroma and a reduces apoptotic response to mutagenic stimuli, which enable it to accumulate progressively more malignant transformations. The resulting granulocytes fail to develop into mature lymphocytes and are released into the circulation, leading to a deficiency in the mature cells and increased susceptibility to infection. ATP-competitive inhibitors of BCR-ABL have been described which prevent the kinase from activating mitogenic and anti-apoptotic pathways (e.g., P-3 kinase and STAT5), leading to the death of the BCR-ABL phenotype cells and thereby providing an effective therapy against CML. The combinations of the present invention useful as BCR-ABL inhibitors are thus especially appropriate for the therapy of diseases related to its overexpression, especially leukemias, such as leukemias, e.g., CML or ALL.

The combinations of the present invention primarily inhibit the growth of blood vessels and are thus, e.g., effective against a number of diseases associated with deregulated angiogenesis, especially diseases caused by ocular neovascularisation, especially retinopathies, such as diabetic retinopathy or age-related macula degeneration, psoriasis, hemangioblastoma, such as haemangioma, mesangial cell proliferative disorders, such as chronic or acute renal diseases, e.g., diabetic nephropathy, malignant nephrosclerosis, thrombotic microangiopathy syndromes or transplant rejection, or especially inflammatory renal disease, such as glomerulonephritis, especially mesangioproliferative glomerulonephritis, hemolytic-uremic syndrome, diabetic nephropathy, hypertensive nephrosclerosis, atheroma, arterial restenosis, autoimmune diseases, diabetes, endometriosis, chronic asthma, and especially neoplastic diseases (solid tumors, but also leukemias and other hematological malignancies), such as especially breast cancer, cancer of the colon, lung cancer (especially small-cell lung cancer), cancer of the prostate or Kaposi's sarcoma. Combinations of the present invention inhibit the growth of tumours and are especially suited to preventing the metastatic spread of tumors and the growth of micrometastases.

We claim:

1. A pharmaceutical combination comprising:
a) a pyrimidylaminobenzamide compound of formula (I); and
b) at least one Flt-3 inhibitor.

2. A pharmaceutical combination according to claim 1, wherein agent a) is selected from 4-methyl-3-[[4-(3-pyridinyl)-2-pyrimidinyl]amino]-N-[5-(4-methyl-1H-imidazol-1-yl)-3-(trifluoromethyl)phenyl]benzamide or salts thereof.

3. A pharmaceutical composition according to claim 2, wherein the Flt-3 inhibitor is selected from N-[(9S,10R,11R,13R)-2,3,10,11,12,13-hexahydro-10-methoxy-9-methyl-1-oxo-9,13-epoxy-1H,9H-diindolo[1,2,3-gh:3′,2′,1′-Im]pyrrolo[3,4-j][1,7]benzodiazonin-11-yl]-N-methylbenzamide; 1-[4-(4-ethyl-piperazinyl-1-ylmethyl)-3-trifluoromethyl-phenyl]-3-[4-(6-methylamino-pyrimidin-4-yloxy-phenyl]-urea; 1-[4-(2-amino-pyrimidin-4-yloxy)-phenyl]-3-[4-(4-ethyl-piperazin-1-ylmethyl)-3-trifluoromethyl-phenyl]-urea; and 1-[4-(2-amino-pyrimidin-4-yloxy)-phenyl]-3-[4-(4-methyl-piperazin-1-ylmethyl)-3-trifluoromethyl-phenyl]-urea.

4. A method for preparing a medicament for the treatment of a proliferative disease comprising a pharmaceutical combination according to claim 1.

5. A method according to claim 4, wherein the proliferative disease is a leukemia.

6. A method according to claim 4, wherein agent a) is selected from 4-methyl-3-[[4-(3-pyridinyl)-2-pyrimidinyl]amino]-N-[5-(4-methyl-1H-imidazol-1-yl)-3-(trifluoromethyl)phenyl]benzamide.

7. A method according to claim 6, wherein the Flt-3 inhibitor is selected from N-[(9S,10R,11R,13R)-2,3,10,11,12,13-hexahydro-10-methoxy-9-methyl-1-oxo-9,13-epoxy-1H,9H-diindolo[1,2,3-gh:3′,2′,1′-1m]pyrrolo[3,4j][1,7]benzodiazonin-11-yl]-N-methylbenzamide; 1-[4-(4-ethyl-piperazinyl-1-ylmethyl)-3-trifluoromethyl-phenyl]-3-[4-(6-methylamino-pyrimidin-4-yloxy-phenyl]-urea; 1-[4-(2-amino-pyrimidin-4-yloxy)-phenyl]-3-[4-(4-ethyl-piperazin-1-ylmethyl)-3-trifluoromethyl-phenyl]-urea; and 1-[4-(2-amino-pyrimidin-4-yloxy)-phenyl]-3-[4-(4-methyl-piperazin-1-ylmethyl)-3-trifluoromethyl-phenyl]-urea.

8. A method for treating or preventing a proliferative disease in a subject in need thereof, comprising co-administration to said subject, e.g., concomitantly or in sequence, of a therapeutically effective amount of at least one Flt-3 inhibitor and a pyrimidylaminobenzamide compound of formula (I).

9. A method according to claim 8, wherein the proliferative disease is a leukemia.

10. A method according to claim 8, wherein the pyrimidylaminobenzamide compound of formula (I) is selected from 4-methyl-3-[[4-(3-pyridinyl)-2-pyrimidinyl]amino]-N-[5-(4-methyl-1H-imidazol-1-yl)-3-(trifluoromethyl)phenyl] benzamide and salts thereof.

11. A method according to claim 8, wherein the Flt-3 inhibitor is selected from N-[(9S,10R,11R,13R)-2,3,10,11,12,13-hexahydro-10-methoxy-9-methyl-1-oxo-9,13-epoxy-1H,9H-diindolo[1,2,3-gh:3′,2′,1′-Im]pyrrolo[3,4j][1,7]benzodiazonin-11-yl]-N-methylbenzamide; 1-[4-(4-ethyl-piperazinyl-1-ylmethyl)-3-trifluoromethyl-phenyl]-3-[4-(6-methylamino-pyrimidin-4-yloxy-phenyl]-urea; 1-[4-(2-amino-pyrimidin-4-yloxy)-phenyl]-3-[4-(4-ethyl-piperazin-1-ylmethyl)-3-trifluoromethyl-phenyl]-urea; and 1-[4-(2-amino-pyrimidin-4-yloxy)-phenyl]-3-[4-(4-methyl-piperazin-1-ylmethyl)-3-trifluoromethyl-phenyl]-urea.

12. A method for treating leukemia comprising administering a combination of:
a) 4-Methyl-3-[[4-(3-pyridinyl)-2-pyrimidinyl]amino]-N-[5-(4-methyl-1H-imidazol-1-yl)-3-(trifluoromethyl)phenyl] benzamide and salts thereof; and
b) a Flt-3 inhibitor.
